(19) United States
(12) Patent Application Publication
(10) Pub. No.: US 2015/0231282 A1
(43) Pub. Date: Aug. 20, 2015

(54) NANOPARTICLE EMULSIONS

(71) Applicant: UNIVERSITY OF WASHINGTON THROUGH ITS CENTER FOR COMMERCIALIZATION, Seattle, WA (US)

(72) Inventors: Lilo Danielle Pozzo, Seattle, WA (US); Matthew O'Donnell, Seattle, WA (US); Thomas J. Matula, Kirkland, WA (US); Kjersta Larson-Smith, Seattle, WA (US); Chen-wei Wei, Seattle, WA (US); Ivan Pelivanov, Seattle, WA (US); Jinjun Xia, Seattle, WA (US)

(73) Assignee: University of Washington through its Center for Commercialization, Seattle, WA (US)

(21) Appl. No.: 14/432,974

(22) PCT Filed: Oct. 1, 2013
(86) PCT No.: PCT/US2013/062896

§ 371 Date: Apr. 1, 2015

Related U.S. Application Data
(60) Provisional application No. 61/708,205, filed on Oct. 1, 2012.

(51) Int. Cl. A61K 49/22; A61M 37/00; A61K 31/28; A61N 5/06; A61K 9/107; A61K 41/00

(52) U.S. Cl. CPC A61K 49/226; A61K 9/107; A61K 41/0052; A61K 31/28; A61N 5/062; A61M 37/0092; A61M 2037/0007

(57) ABSTRACT

Composites formed from a liquid core encapsulated by a plurality of nanoparticles are provided herein. The composites in certain embodiments are droplets comprising a hydrophobic dispersed phase within a hydrophilic continuous phase, thereby forming an emulsion. The composites can be used as contrast agents for imaging, therapeutic agents, and adapted for other uses according to the unique properties of the composites disclosed herein.

### CROSS-REFERENCE TO RELATED APPLICATION

This application claims the benefit of U.S. Provisional Application No. 61/708,205, filed Oct. 1, 2012, the disclosure of which is incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is: 43071_Seq_Listing_Final 20131001.txt. The file is 1 KB; was created on Oct. 1, 2013; and is being submitted via EFS-Web with the filing of the specification.

### BACKGROUND

Emulsions are inherently unstable and must be stabilized against coalescence with suitable emulsifying agents such as surfactants, polymers or particles. Surfactants are widely used emulsifiers because they adsorb strongly to oil-water interfaces with hydrophobic parts pointed towards in the oil phase and their hydrophilic sections pointed towards the water phase. This causes a decrease in the interfacial energy and also hinders dilation and droplet coalescence. Similarly, block copolymers have been shown to be effective stabilizers because the chains are able to penetrate the oil-water interface to reduce the interfacial tension and prevent emulsion destabilization.

Interfaces can also be stabilized by colloidal particles forming dispersions known as Pickering emulsions. While particle-stabilized emulsions have been widely studied for several decades, recently there is renewed interest in these materials for many new applications such as the preparation of colloidosomes and composite particles. Colloidosomes are porous microcapsules that are constructed from a composite shell of particles. These structures can be used to encapsulate proteins or pharmaceuticals for applications in controlled drug-delivery. The most useful colloidosomes in these applications have sizes on the order of just a few microns in diameter. Therefore, nanoparticles are used as the building blocks for these structures. Furthermore, the permeability of the colloidosome could be tuned with particle size, making the structures useful for controlled delivery of small molecule drugs. Additionally, colloidosomes prepared from plasmonic nanoparticles could find applications in photothermal therapy and in photoacoustic imaging applications. Currently, gold nanoshells show remarkable promise in photothermal cancer therapy because of their tunable plasmon resonance in the near-infrared (NIR) wavelength region where blood and tissue are most transmissive. When illuminated, the nanoshells serve as a localized heat source, photothermally inducing cell death in targeted tissues. While these nanoshells are very effective, they are typically 100-200 nm in diameter, which is too large for clearance via the renal system.

Despite the benefits of the colloidal dispersions described above, further improvements in dispersion technology are desirable in order to improve biological compatibility and expand the functional utility of the dispersions.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, an emulsion is provided. In one embodiment, the emulsion includes:

a hydrophilic continuous phase; and

a hydrophobic dispersed phase comprising a plurality of droplets, the droplets comprising a composite, comprising:
a core comprising a hydrophobic liquid;
and a plurality of nanoparticles substantially encapsulating the core, wherein the plurality of nanoparticles are associated with a plurality of emulsifier molecules.

In one aspect, a method of therapy is provided. In one embodiment, the method includes the steps of contacting a biological tissue with a composite according to any of the embodiments disclosed herein; and applying energy to the biological tissue.

In one aspect, a method of imaging is provided. In one embodiment, the method includes the steps of providing a composite according to the embodiments disclosed herein; applying energy to the composite; and detecting a signal emitted from the composite.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, which will now be described.

FIGS. 1A and 1B schematically illustrate emulsions in accordance with the disclosed embodiments having a core comprising a hydrophobic liquid encapsulated by a plurality of nanoparticles.

FIG. 2. Schematic representation of the synthesis of nanoparticle surfactants and the stabilized emulsions. Particles are functionalized with a controlled amount of PEG-thiol (represented in blue) followed by saturation with a hydrophobic alkane-thiol (represented in red). Clusters spontaneously form in water dispersions. After emulsification with oil, nanoparticles adsorb to the oil-water interface with the alkane-functionalized portion of the particle exposed towards the oil phase.

FIGS. 3A-3D. Micrographs of a 0.01 vol % hexadecane in water emulsion stabilized by 0.8 PEG chains/nm² Au and butane-thiol functionalized amphiphilic gold nanoparticles taken with FIG. 3A optical microscopy, FIG. 3B scanning electron microscopy and FIG. 3C transmission electron microscopy. FIG. 3D Characteristic particle cluster observed with transmission electron microscopy.

FIGS. 4A-4C. FIG. 4A UV-Vis absorption spectra of gold nanoparticles functionalized with 0.8 PEG chains/nm² Au before and after butane-thiol functionalization and after emulsification with hexadecane. FIG. 4B Photograph demonstrating the color shift of PEG-functionalized singlet particles, clustered nanoparticle surfactants and emulsions stabilized by the nanoparticle surfactants. FIG. 4C Photographs of a concentrated particle-stabilized emulsion with light transmitted through and reflected.

FIG. 5. Desmeared SAXS data for bare gold nanoparticles, clustered amphiphilic gold nanoparticles in water and emulsified amphiphilic gold nanoparticles. The bottom curve is scattering from bare gold particles in water which is fit by a spherical model with Rₚ=6.11 nm, φₚ=0.00024 ρ_{w}=9.46*10⁻⁶ Å⁻² and ρₚ=1.25*10⁻⁴ Å⁻². The middle curve (shifted by a factor of 10 for clarity) is the scattering data for a nanoparticle surfactant cluster in water and the modeled scattering curve calculated using the Debye equation. The top curve (shifted by a factor of 10⁴) is for a 1.0 vol % hexadecane in water emulsion stabilized by 0.024 vol % 6.11 nm amphiphilic gold particles. Parameters of the model are Rₒ=494 nm with a polydispersity index of 0.1 (Gaussian distribution), Rₚ=6.11 nm, δ=0, φₒ=0.00998, φₚ^{T}=0.00024, ρ_{w}=9.46*10⁻⁶ Å⁻², ρₒ=7.52*10⁻⁶ Å⁻², ρₚ=1.25*10⁻⁴ Å⁻², 88.0% interface coverage.

FIGS. 6A and 6B. FIG. 6A UV-Vis spectra of 1 vol % hexadecane in water emulsions stabilized by nanoparticle surfactants with 0.6 PEG chains/nm² Au and different alkane-thiols. The curves are normalized by the total particle concentration in each sample. FIG. 6B Absorbance peak plotted as a function of PEG-thiol concentration for butane-thiol (circles), octane-thiol (triangles) and dodecane-thiol (squares) where the dotted line is the absorbance for bare gold particles.

FIGS. 7A and 7B. FIG. 7A SAXS data and fits to the Pickering emulsion model for 1 vol % hexadecane in water emulsions stabilized by surfactant nanoparticles containing 1.8 PEG chains/nm² Au and different types of alkane-thiol molecules. Parameters of the model are Rₒ=498 nm with a polydispersity index of 0.1 (Gaussian distribution), Rₚ=6.55 nm, δ=0, φφₒ=0.0998, φₚ^{T}=0.00024, ρ_{w}, =9.46*10⁻⁶ Å⁻², ρₒ=7.52*10⁻⁶ Å⁻², ρₚ=1.25*10⁻⁴ Å⁻². The butane-thiol emulsion (shifted by a factor of 10³), octane-thiol emulsion (shifted by a factor of 10²), dodecane-thiol emulsion (shifted by a factor of 10) and no alkane-thiol emulsion are best fit with a 85.5%, 60.0%, 34.6% and 0% interface coverage respectively. FIG. 7B Percent interface coverage plotted as a function of PEG-thiol concentration for butane-thiol (circles), octane-thiol (triangles) and dodecane-thiol (squares).

FIG. 8A schematically illustrates the transition of a composite in accordance with the provided embodiments from a liquid core to a gas core, and optionally back from a gas to a liquid core.

FIG. 8B shows an example of an experimental setup for stimulating and imaging the composite contrast agents of the present disclosure, including a tube containing the samples, a pulsed optical source, a linear array transducer for delivering US pulses, and a wideband PVDF (polyvinylidene fluoride) transducer to receive the acoustic signals.

FIG. 9 presents experimental results for four different composite contrast agents (four rows). The left column presents images for US-alone probing. The central column presents images for optical probing alone (PA). The right column shows images for simultaneous US+PA probing of the system. The first row is for only gold nanoparticles not integrated into an emulsion—the response here should be nearly identical to stable background signals encountered in the body. The remaining three rows are for emulsions composed of a perfluorohexane oil core coated with gold nanoparticles with different surface coatings and optical absorption.

FIG. 10 presents acoustic spectra for all the images of FIG. 9. Notice the broader spectrum for the butane system suggesting the presence of non-linearities.

FIG. 11 presents a set of subtracted images computed from the raw images of FIG. 9. Note the strong signal for the (US+PA)-US−PA image for the butane system. This suggests either a strong non-linear response or a modulation of the PA source for this nanosystem. In either case, this signal can be easily distinguished from background sources that will act more like the pure gold system shown in the top row. These results demonstrate that the composite nanosystem combining an emulsion droplet core with, for example, gold nanospheres at the interface can be used to enhance the specific contrast of molecular images obtained by processing integrated US+PA probes.

FIG. 12 shows the spectra of the subtracted images of FIG. 11. The broader spectra of the subtracted signals of the butane system can be clearly seen. By choosing a higher sub-band (1.5 MHz to 20 MHz), the contrast of the subtracted image of emulsion droplets with gold nanospheres (GNSs) over pure GNSs can increase from 12.8 dB (full band, 0.05-30 MHz) to 15.7 dB. Again, the enhanced contrast using sub-band imaging provides the evidence of generation of non-linear signals of the emulsion droplets.

### DETAILED DESCRIPTION

Composites formed from a liquid core encapsulated by a plurality of nanoparticles are provided herein. The composites in certain embodiments are droplets comprising a hydrophobic dispersed phase within a hydrophilic continuous phase, thereby forming an emulsion. The composites can be used as contrast agents for imaging, therapeutic agents, and adapted for other uses according to the unique properties of the composites disclosed herein.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of skill in the art, such as the Oxford Dictionary of Biochemistry and Molecular Biology (Ed. Anthony Smith, Oxford University Press, Oxford, 2004).

As used herein and unless otherwise indicated, the terms “a” and “an” are taken to mean “one,” “at least one,” or “one or more.” Unless otherwise required by context, singular terms used herein shall include pluralities and plural terms shall include the singular.

Nanoparticle Composites

In one aspect, an emulsion is provided. In one embodiment, the emulsion includes:

a hydrophilic continuous phase; and

a hydrophobic dispersed phase comprising a plurality of droplets, the droplets comprising a composite, comprising:
a core comprising a hydrophobic liquid;
and a plurality of nanoparticles substantially encapsulating the core, wherein the plurality of nanoparticles are associated with a plurality of emulsifier molecules.

The creation of emulsions is well known to those of skill in the art, as is the composition of an emulsion having a hydrophilic continuous phase and a hydrophobic dispersed phase. The hydrophilic continuous phase of the provided embodiments can be any hydrophilic liquid known to those of skill in the art. Water is an exemplary hydrophilic continuous phase, although the provided emulsions are not limited to such. Non-aqueous continuous phases may also be used.

The hydrophobic dispersed phase comprises a plurality of droplets. The droplets comprise a composite. The composite comprises a core, comprising a hydrophobic liquid, and a plurality of nanoparticles substantially encapsulating the core. As used herein, in certain embodiments the term “substantially encapsulating” refers to an encapsulation degree (i.e., percentage of the surface of the core covered by the nanoparticles) sufficient to provide the required effects (e.g., optical effects). In one embodiment, the term “substantially encapsulating” refers to an encapsulation degree of greater that 25%. In one embodiment, the term “substantially encapsulating” refers to an encapsulation degree of greater that 50%. In one embodiment, the term “substantially encapsulating” refers to an encapsulation degree of greater that 75%. In one embodiment, the term “substantially encapsulating” refers to an encapsulation degree of 100%.

The plurality of nanoparticles are associated with a plurality of emulsifier molecules.

The emulsifier molecules associated with the nanoparticles provide a functionalized nanoparticle encapsulant to the hydrophobic liquid core. The combined properties of the core, nanoparticles, and emulsifier molecules, allows for the hydrophobic dispersed phase to be supported in the hydrophobic continuous phase as an emulsion.

The core comprises a hydrophobic liquid. In one embodiment, the hydrophobic liquid is selected from the group consisting of hydrocarbon oils, fluorinated oils, and combinations thereof. Hydrocarbon oils are well known to those of skill in the art, and any such hydrocarbon oil can be used as long as the properties of the hydrocarbon oil allow for the formation of the provided composites. Notable hydrocarbon oils include hexane, dodecane, cyclopentane, cyclohexane, and hexadecane. In one embodiment, the hydrocarbon oil is a homogenous hydrocarbon containing only carbon and hydrogen.

Fluorinated oils are also well known to those of skill in the art, and equally numerous as hydrocarbon oils. Representative fluorinated oils include fluorinated analogs of the hydrocarbons disclosed above. Exemplary fluorinated oils include perfluoropentane, perfluorohexane, perfluoroheptane, perfluorodecalin, and perfluoro(methylcyclohexane).

It will be appreciated that other fluorinated oils can be used in the provided composites, as long as the fluorinated oils have the properties required for the applications in which the composites are used (e.g., proper boiling point, viscosity, hydrophobic nature, etc.).

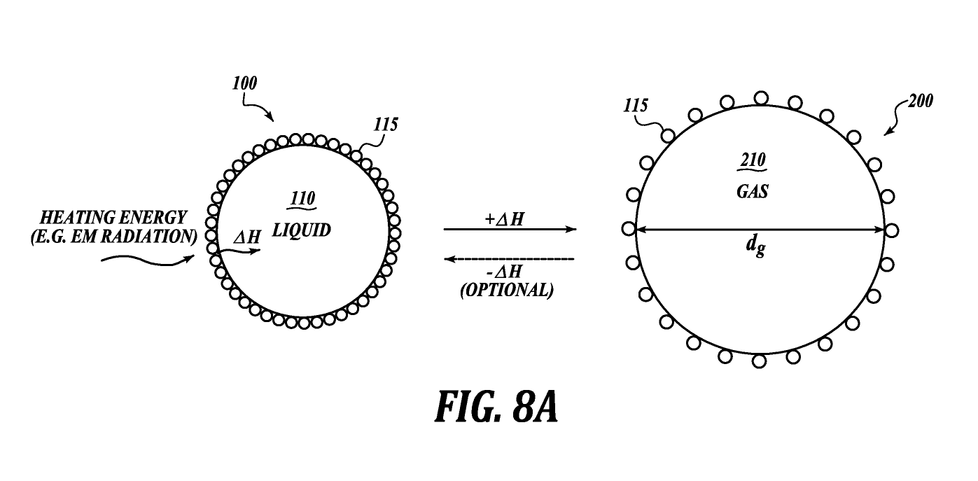

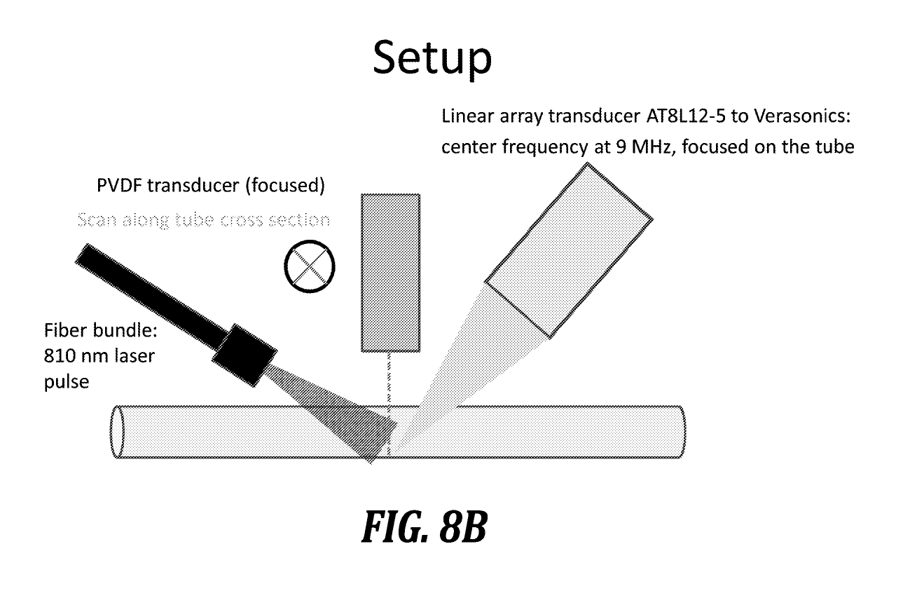

In one embodiment, the hydrophobic liquid is capable of vaporization when heated to a temperature from 20° C. to 80° C. As will be discussed further herein, the composites in certain applications are heated using electromagnetic radiation so as to expand the liquid of the core to provide a gas core surrounded by nanoparticles (see FIG. 8A). In order to facilitate the use of the composites as a contrast agent in biological applications, the vaporization temperature must be relatively low, so as to not harm living subjects in which the compositions are applied. Therefore, the vaporization temperature of the hydrophobic liquid in certain embodiments is below 100° C. In other embodiments, the vaporization temperature is from 30° C. to 70° C. In another embodiment, the vaporization temperature is from 40° C. to 60° C.

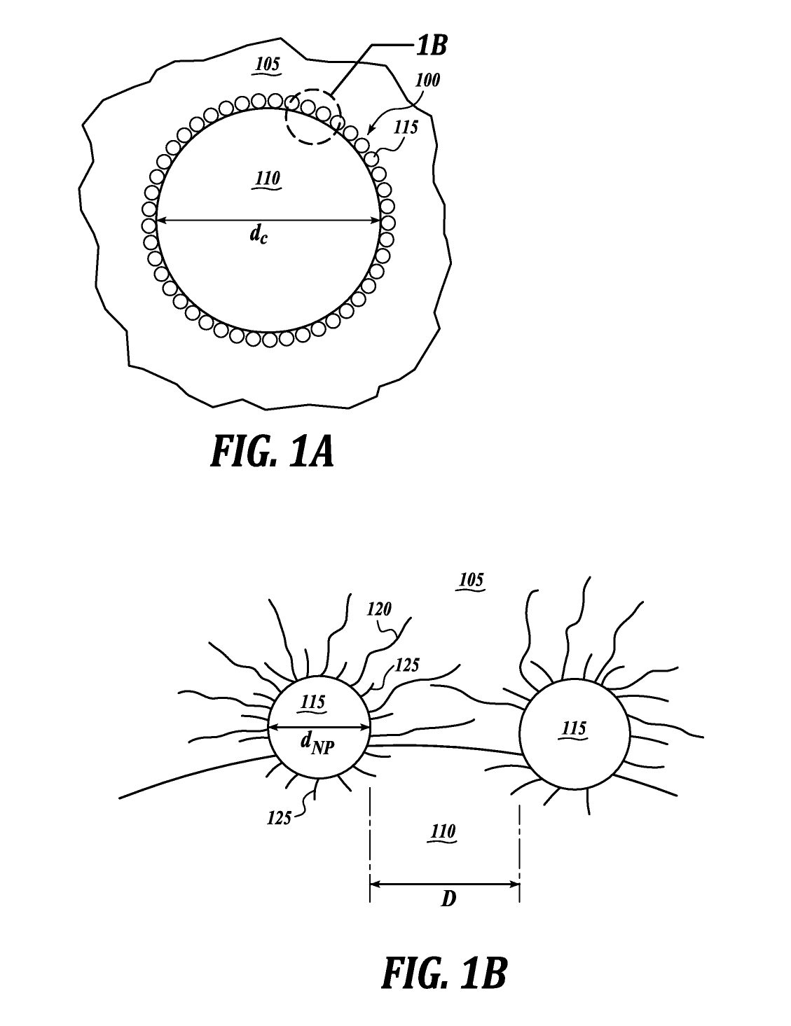

The compositions provided herein may be better understood with reference to FIGS. 1A and 1B. Referring to FIG. 1A, a single droplet 100 within an emulsion is illustrated. The emulsion includes a hydrophilic continuous phase 105 and the droplet 100 representing a hydrophobic dispersed phase. The droplet 100 includes a core 110 comprising a hydrophobic liquid, encapsulated by a plurality of nanoparticles 115.

The core 110 has a diameter indicated in FIG. 1A as d_{c}. The core is defined as having an average diameter due to the typical polydispersity found in the produced emulsions. Therefore, the size of the core may vary from droplet to droplet, although the average diameter of the core is within the provided range. It will be appreciated that monodisperse emulsions are also contemplated. Therefore, for a monodisperse emulsion, the diameter of the core is not an average diameter, but the uniform diameter of the cores of the droplets of the emulsion.

In one embodiment, the core has an average diameter of from 5-500 nm. In one embodiment, the core has an average diameter of from 5-200 nm. In one embodiment, the core has an average diameter of from 5-100 nm. In one embodiment, the core has an average diameter of from 50-200 nm. When the composites are used in certain embodiments of the methods disclosed herein, a relatively small core is preferred, as will be described in more detail below.

FIG. 1B is a close-up view of a portion of FIG. 1A, that includes two metal nanoparticles 115 situated on a surface of the core 110. The nanoparticles 115 have a diameter designated as d_{NP}. The nanoparticles 115 are separated by a distance indicated as D.

While the nanoparticles 115 are illustrated in the FIGURES and described primarily herein as being spheres (e.g., “nanospheres”), it will be appreciated that the disclosed embodiments are not limited to only spherical nanoparticles. Any nanoparticle shape that produces the required effects (e.g., optical properties, size range, and ability to form the emulsions) can be utilized. Additional exemplary shapes include pyramids, cubes, other polygons, rods, and irregular shapes.

The nanoparticles 115 are associated with a plurality of emulsifier molecules, illustrated in FIG. 1B as 120 and 125, two different types of emulsifier molecules. While FIG. 1B illustrates two different types of emulsifiers molecules 120 and 125, it will be appreciated that a single type of emulsifier molecule is also contemplated, as well as the use of three or more types of emulsifier molecules. The emulsifier molecules must be capable of being associated with (e.g., bound to) the nanoparticles 115, as well as provide for emulsification of the droplets in the hydrophobic continuous phase. In one embodiment, the emulsifier molecules render the nanoparticles amphiphilic. Accordingly, for the nanoparticles to be amphiphilic, the emulsifier molecules must provide both hydrophobic and hydrophilic functionality. As illustrated in FIG. 1B, the amphiphilic functionality is provided by using two different types of emulsifier molecules 120 and 125, wherein, for example, emulsifier molecule 120 is a relatively large hydrophobic molecule, such as polyethylene glycol (PEG) and the second emulsifier molecule is a shorter hydrophobic molecule, such as an alkane. Therefore, in one embodiment, the emulsifier molecules comprise both hydrophobic molecules and hydrophilic molecules. In another embodiment, the emulsifier molecules are amphiphilic molecules. As such, single molecules having amphiphilic functionality are known to those of skill in the art and can be incorporated into the present compositions.

Representative amphiphilic molecules include PEG-polystyrene block copolymers functionalized with a thiol (or other appropriate) end group or PEG ligands functionalized with alkane thiols. Dojindo Molecular Technologies, Inc. (Japan) sells several representative amphiphilic molecules according to these embodiments.

In one embodiment, the amphiphilic molecules are peptides functionalized to bind to the nanoparticles (e.g., thiol-functionalized peptides for binding to gold nanoparticles). An exemplary sequence is thiol-AAAAAKKKKKK [SEQ ID NO:1] with thiol functionality in the N-terminus. Any number of sequences could be used, containing a series of hydrophobic amino acids (A, V, I, L, M, F, Y or W), close to the thiol functional group, and one or more hydrophilic amino acids (R, H, K, D, E, S, T, N, Q) at the opposite end. Alternatively, cysteine could be used instead of a terminal thiol group because it has metal binding functionality.

In one embodiment, the emulsifier molecules are not polymers. As used herein, a polymer is defined as any molecule having more than 10 monomer units. Certain polymers are known to be amphiphilic, although the preferred embodiments of the composites do not include polymers, but instead incorporate relatively small molecules that provide the nanoparticles with amphiphilic properties.

In one embodiment, the emulsifier molecules are surfactant molecules. As used herein, the term surfactant is used to describe molecules having both hydrophobic groups and hydrophilic groups. As such, surfactants are well known to those of skill in the art as finding use in emulsions. Therefore, surfactants known to those of skill in the art are contemplated for use in the provided embodiments.

In one embodiment, the emulsifier molecules are selected from the group consisting of thiol-terminated polyethylene glycols, thiol-terminated straight chain alkyl molecules, thiol-terminated branched alkyl molecules, thiol-terminated perfluorocarbons, and combinations thereof. It will be appreciated that this is only a representative list, and other emulsifier molecules can be used, so long as the emulsions can be stabilized using the emulsifier molecules.

While thiol-functionalized emulsifier molecules are primarily disclosed herein, it will be appreciated that any functionality allowing the emulsifier molecules to bind to (or become associated with) the nanoparticles are contemplated. Functional groups analogous to thiols include phosphonates and disulfides.

In one embodiment, the emulsifier molecules are charged molecules. Charged molecules can be used instead of surfactant-type molecules in order to provide the necessary emulsification. Most hydrophobic fluids will form a negative surface charge when forming an interface with water. The reason, is the orientation of water molecules and the preferential adsorption of OH— groups. This is strongly pH-dependent. In the present embodiments, the nanoparticles will have to be positively charged. This can be accomplished with a functional group associated with the nanoparticles, such as an alkane thiol with an amine end-group. Peptides with cationic (positive) amino acids can also be used.

Representative charged molecules include mercaptoundecyl-trimethylammonium bromide, 6-Amino-1-hexanethiol hydrochloride, 11-Aminoundecanethiol hydrochloride, (3-Mercaptopropyl)ammonium chloride, cysteamine, and thiol-terminated cationic peptides.

In one embodiment, the emulsifier molecules are covalently bound to the nanoparticles. Additional means of associating the emulsifier molecules with the nanoparticles are also contemplated, including ionic bonding, Van der Waal's forces, physical trapping (e.g., entanglement), and other methods known to those of skill in the art. However, from a practical perspective, covalently bound emulsifier molecules provide great flexibility with regard to the attachment of the emulsifier molecules to the nanoparticles.

The nanoparticles of the composite can be formed from any material known to those of skill in the art, as long as the necessary properties of the composite, and uses thereof, are satisfied. In this regard, the nanoparticles must be able to enable the formation of the hydrophobic disbursed phase by substantially encapsulating the core and associate with a plurality of emulsifier molecules.

In one embodiment, the nanoparticles comprise a biologic targeting agent. Biologic targeting agents are well known to those of skill in the art and can be used to functionalize the nanoparticle so as to associate with a particular biologic target. Representative biologic targets include antibodies, aptamers, peptides, and proteins. Any biologic targeting agents known to those of skill in the art can be used with the provided embodiments as long as the biologic targeting agent can be associated with (e.g., bound to) the nanoparticles.

With reference to FIG. 1B, the nanoparticles 115 have a diameter (d_{NP}), from 3 nm to 20 nm. In certain embodiments, the nanoparticles are monodisperse, and therefore, the noted diameter range is for all nanoparticles of the composite. In other embodiments, the nanoparticles are polydisperse, and therefore, the recited diameter range is an average diameter range across the population of nanoparticles in the composite. In other embodiments, the nanoparticles have a diameter of from 3 nm to 10 nm. In other embodiments, the nanoparticles have a diameter of from 3 nm to 6 nm. Finally, in another embodiment, the nanoparticles have a diameter of 5 nm or less. For biological applications intended for use with mammals (e.g., humans), nanoparticles with a diameter (or largest dimension for non-spherical nanoparticles) of 5 nm or less so as to be able to be cleared by the renal system. Larger nanoparticles may not be cleared properly and therefore may pose a health hazard.

Referring again to FIG. 1B, the plurality of nanoparticles may be separated from neighboring nanoparticles by a particular distance, D. In certain embodiments, the distance D is less than 2 times the diameter, d_{NP}, of the nanoparticles. In certain embodiments, the distance D is less than the diameter of the nanoparticles. Characterized in another way, in another embodiment, each of the plurality of nanoparticles are separated from adjacent nanoparticles by a distance that is less than 2 nm. In one embodiment, a majority of the plurality of nanoparticles are in contact with at least one other nanoparticle. Conversely, in other embodiments, the nanoparticles do not touch, yet are in close proximity (e.g., if the nanoparticles are spread in a network across the surface of the core 110).

In one embodiment, the nanoparticles are selected from the group consisting of metal nanoparticles, magnetic nanoparticles, ferroelectric nanoparticles, and semiconductor nanoparticles. In certain embodiments that will be discussed at length herein, the nanoparticles are metal nanoparticles. In further embodiments, the metal nanoparticles are selected from the group consisting of gold, silver, copper, palladium, and platinum nanoparticles. Metal nanoparticles are particularly useful in the disclosed embodiments due to the presence of plasmon resonance activity on the surface of the metallic nanoparticles, which gives rise to optical properties (e.g., absorption of electromagnetic radiation) that is tunable based on the size of the metal nanoparticles, as well as the proximity of the nanoparticles to each other.

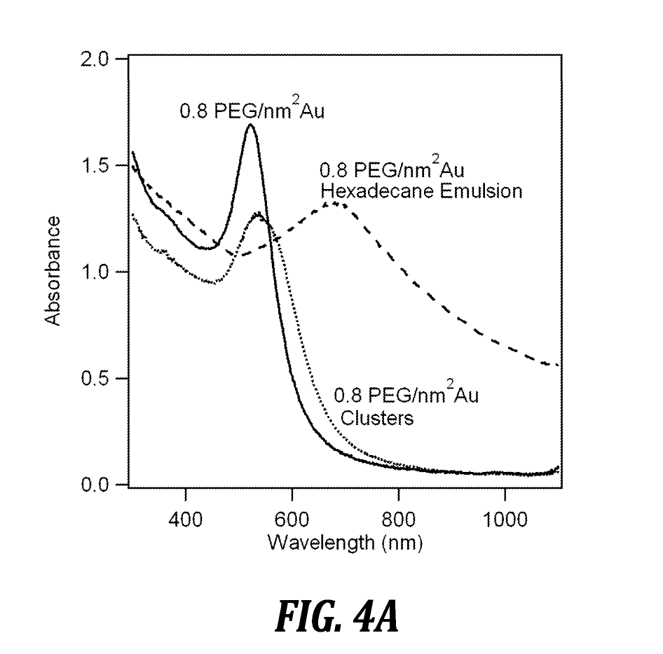

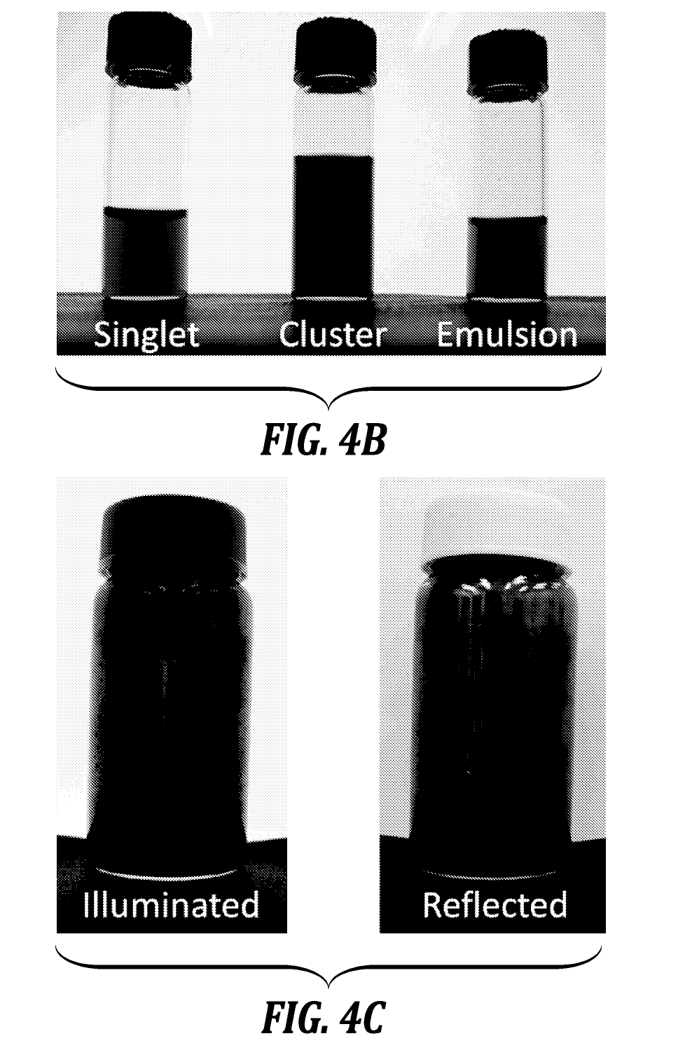

With regard to the absorptivity of nanoparticles, in one embodiment, each individual nanoparticle within the plurality of nanoparticles has an individual absorption peak and the plurality of nanoparticles has a collective absorption peak that is at a longer wavelength than the individual absorption peak. This effect is referred to herein as “red-shifting” of the optical absorption spectrum of the nanoparticles. Based on the proximity effect of nanoparticles disposed within close proximity to other nanoparticles, the optical properties of the nanoparticles can be used to tune the absorption of the collective plurality of nanoparticles to a longer (i.e., lower energy) wavelength than would be absorbed by a single nanoparticle. As will be described further in the examples below, particularly with regard to FIG. 4A, experimental evidence is presented demonstrating the red-shifting of emulsions of hexadecane-gold nanoparticle emulsions compared to individual gold nanoparticles. As illustrated in FIG. 4A, the red-shifting is on the order of 200 nm for the exemplary composition of the gold nanoparticle emulsion. Such red-shifting of the absorbance wavelength of nanoparticles provides a particular benefit when the disclosed composites are used for applications with living tissue, which is transparent in certain near-infrared (NIR) wavelengths, but opaque at visible wavelengths. By red-shifting the absorbance of the nanoparticles into the NIR spectrum, low energy NIR radiation can be used to heat the nanoparticles (for applications that will be discussed later) efficiently and with minimal absorption of the radiation by the tissue.

With regard to the absorption wavelengths of the nanoparticles, in one embodiment, the absorption of the nanoparticles is in the range of between 500 nm to 1500 nm. In another embodiment, the absorption of the nanoparticles is from 1000 nm to 1500 nm. Finally, in another embodiment, the absorption of the nanoparticles is in the near-infrared range of the spectrum. As used herein, the term “absorption” as it relates to the nanoparticles refers to the peak absorption, although it will be appreciated by those of skill in the art that absorption peaks can be broad, covering several hundred nanometers of spectrum in certain cases, however, the values recited herein relate to the peak absorption wavelengths of the nanoparticles.

In one embodiment, the nanoparticles are configured to heat the core when irradiated with electromagnetic radiation. Particularly, the nanoparticles can be configured to absorb electromagnetic radiation and transfer the electromagnetic radiation into heat, thus heating the liquid of the core 110. As illustrated in FIG. 8A, the core 110 is in a liquid state prior to irradiation, but expands to a gaseous (or vapor) state 210 when sufficient heat is applied to the liquid core 110 via the nanoparticles 115. A bubble 200 is then formed. It will be appreciated that other sources of energy may be used to heat the liquid core, such as sonic, ultrasonic, mechanical, and other means known to those of skill in the art.

The bubble 200 has a core 210 that is generally referred to herein as containing a gas, or in a gaseous state. However, the core 210 may also have a vapor, as opposed to gaseous, state. Both states provide a bubble 200, with the distinction of the provided embodiments being that the “vapor” state can possibly condense into the liquid core 110. As used herein, the “gaseous” state will not condense and the bubble 200 may dissolve. Generally, the descriptions provided herein use the “gas” or “gaseous” terminology, although it will be appreciated that these embodiments can also have a “vapor” core 210, unless specifically noted otherwise.

Referring once again to FIG. 8A, the heat applied to the droplet 100, which is illustrated as a AH, heats the liquid core 110 sufficiently to vaporize it and transform it into a gas core 210 so as to form a bubble 200 that includes nanoparticles 115 disbursed across the surface of the bubble 200. The diameter of the core 110 expands from the diameter illustrated in FIG. 1A to the larger diameter, d_{g}, illustrated in FIG. 8A. Comparing d_{c} to d_{g}, d_{c} is always smaller.

In certain embodiments, when the liquid core 110 is transformed into a gaseous core 210, the change is permanent and the composite remains a bubble composite 200. In other embodiments, when sufficient energy is removed from the system (−ΔH), the gas core 210 condenses to form a liquid core 110, thereby reverting back to the original liquid composite 110. The properties of the hydrophobic liquid of the core, as well as the heating applied to the core, and other factors, may determine whether the bubble composite 200 will condense back into the liquid composite 100. Typically, if sufficient energy is provided to the core 110, the change to a gas core 210 will be permanent. Without sufficient energy, the change will be temporary and condensation back to the liquid core 110 will occur.

Therefore, in one embodiment, the disbursed phase maintains structural integrity when the hydrophobic liquid changes states to a gas. Specifically, the nanoparticles 115 remain on the periphery of the gas core 210 when a bubble 200 is formed, thereby maintaining the spherical structure and partial encapsulation of the gas core 210 by the nanoparticles 115. If condensation to the liquid droplet 110 occurs, the nanoparticles may return to their previous confirmation.

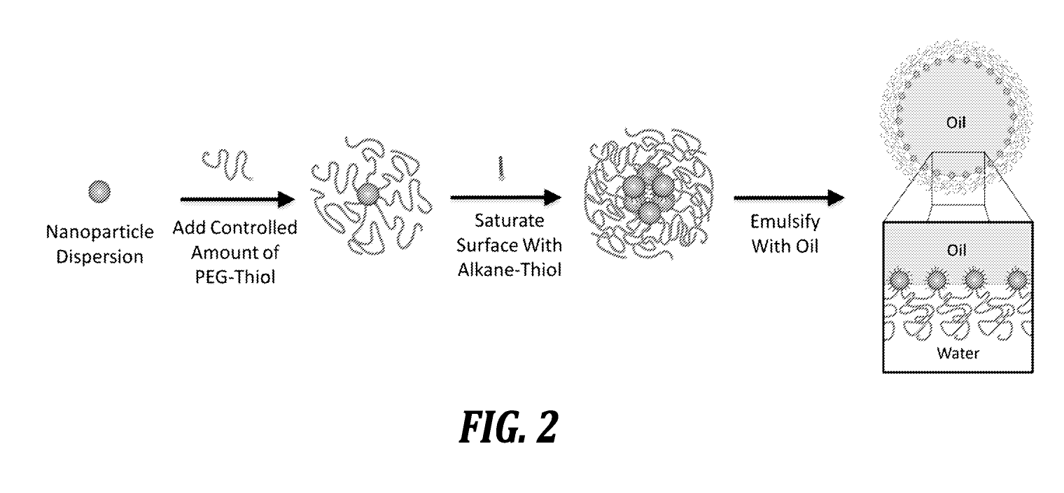

Example 1 below, as well as FIGS. 2-7B described therein, describe exemplary embodiments of the emulsions, particularly with regard to gold nanoparticles encapsulating hydrocarbon oil droplets disbursed in an aqueous phase.

Methods of Treatment Using the Composites

In one aspect, a method of therapy is provided. In one embodiment, the method includes the steps of contacting a biological tissue with a composite according to any of the embodiments disclosed herein; and applying energy to the biological tissue.

One shortcoming of present optical therapies is that for sufficient energy to be applied to the agents within a subject, visible wavelengths must be used. These wavelengths are absorbed by the tissue and therefore reduce the penetration depth of the optical signal to a few millimeters. The present composites can be designed to absorb in the NIR region where tissue is transparent. Because longer wavelengths penetrate further into tissues, the penetration depth can be increased by an order of magnitude. This allows for access to an entire new set of tissue within a body that can be analyzed using optical methods. For example, peripheral vessels lying one to five centimeters deep from the body surface can be accessed optically with sufficient light intensities for both diagnostic and therapeutic applications.

The composites disclosed herein have the ability to change from a liquid core 110 to a gaseous core 210 and form a bubble 200, as described above with reference to FIG. 8A. This expansion, and possible contraction, of the composites allows for the composites to perform mechanical work based on energy applied to the composite. The expansion and contraction of the composites can be used for therapeutic benefits in any manner known to those of skill in the art. Particular exemplary embodiments will be disclosed herein, although it will be appreciated that the expansion and contraction of the composites can be used to perform mechanical work in any situation wherein the composites are disposed in close proximity to a surface or tissue upon which work is to be performed.

Applying energy to the composite will expand the composite to form a bubble 200, thereby applying mechanical work to the surface or tissue. If the bubble 200 then contracts to a droplet 100, the process can be repeated and the mechanical work of the expansion and contraction process repeatedly applied to the tissue or surface.

The biological tissue can be any tissue upon which mechanical work is desired to be exerted. Representative biological tissues include blood clots, atherosclerotic plaques, tumors, fat cells, fibroids, and moles. This list is exemplary and non-exhaustive.

The embodiments of this aspect of this method rely on the expansion of the droplet 100 to a bubble 200, as illustrated in FIG. 8A. The expansion is effected by applying energy to the tissue. The energy can be in the form of electromagnetic radiation, sonic or ultrasonic energy, or other energy sources known to those of skill in the art. The energy is sufficient to result in a phase transition of the core, such as the liquid core 110 transition to a gaseous core 210 in FIG. 8A. In certain embodiments, the process is reversible, and the gas core 210 returns to the liquid phase to form a liquid core 110 after the energy is no longer applied to the biological tissue. Alternatively, instead of reversing the expansion to a gas, applying the energy may cause the composite to substantially break apart, thereby effectively destroying the composite without performing a single transition from liquid to gas and back. In other embodiments, the composite transitions back and forth between liquid and gas a plurality of times, but eventually breaks apart.

In one embodiment, applying energy to the tissue results in thermally exciting the plurality of nanoparticles. By thermally exciting the plurality of nanoparticles, energy is transferred from the energy source (e.g., electromagnetic energy source) to the liquid core 110. With sufficient thermal excitation of the core 110, the liquid expands to a gas in order to form the bubble 200. As noted above with regard to the composites, the absorption wavelength of the metal nanoparticles can be used to deliver energy to the composites so as to transform the energy into thermal energy and heat the liquid core 110.

When the composites are used in the methods disclosed herein, in certain embodiments a relatively small core (e.g., less than 200 nm) is preferred. A smaller core allows for more efficient heating of the core so as to vaporize the core liquid to create a bubble 200. This efficiency is partly due to an increase of surface area per volume, which allows for more nanoparticles on the core per unit of volume; this in turn allows for greater heating efficiency. Additionally, the smaller the core, the less energy is required to vaporize the core liquid.

In one embodiment, the applied energy is sonic energy. Sonic energy can be used to excite the metal nanoparticles and/or the liquid core 110 so as to provide the necessary heating required to form the bubble 200.

In one embodiment, both electromagnetic and sonic energy are applied simultaneously to the composite. The electromagnetic and sonic energy can both be used to provide the required heating, or can be used to perform different functions. In one embodiment, the electromagnetic energy impinges on the biological tissue at substantially the same time as peak negative pressure induced by the sonic energy. The peak negative pressure serves to reduce the threshold for the phase transformation, making it easier (less energy needed) to transform from a liquid to a gas.

When the composites are expanded and contracted in order to apply mechanical work to tissue, in certain embodiments, the phase change and mechanical work ablates or otherwise destroys the biological tissue. Such an application is akin to repeatedly hitting the tissue using the expansion of the composite into a bubble 200. Mechanical work can be applied repeatedly to the same biological tissue in order to ablate or otherwise destroy it.

In another application, the composites can be used for acoustically enhanced diffusion. In such an application, the method further includes a step of applying energy to the biological tissue further includes the steps of applying electromagnetic energy at a first time sufficient to cause a phase change in the core; and applying sonic energy at a second time sufficient to urge the composite through the biological tissue. In embodiments such as this, the electromagnetic energy is used to cause the phase change from droplet 100 to bubble 200, which then increases susceptibility of the composite to physical movement resulting from the applied sonic energy. Therefore, the sonic energy impinges upon the bubble 200 in order to drive the bubble 200 further towards tissue, or in a particular direction. If the bubble transitions back to a droplet 100, in certain embodiments the sonic energy no longer has sufficient impact on the composite to produce movement. If the bubble 200 transitions back to a droplet 100 and sonic energy continues to be applied, the droplet 100 will not be driven in the direction of the sonic energy travel. However, if electromagnetic energy is again applied to the droplet 100, and expansion of the droplet 100 into the bubble 200 is effected again, the sonic energy will once again effect movement of the bubble in the direction of the sonic energy travel (towards biological tissue). Therefore, a ratchet-like motion can be achieved by applying sonic energy in conjunction with expansion and contraction of the composite.

In yet another application, the composites can be used for targeted thermal treatment. Because the composites can be formed to absorb at NIR wavelengths, efficient heating within a subject can be performed using only low energy electromagnetic radiation. Such targeted heating can be used in various medical applications known to those of skill in the art. Using the provided composites, an additional degree of control over the thermal treatment can be realized. The composites can be used as indicators for when the environment around the composites reaches a certain temperature. That is, if the droplets transform into bubbles at a specific temperature, the temperature of any therapy (e.g., radiation therapy or focused ultrasound) can be monitored in situ because one can monitor when the droplets transform. Thus, an in-situ temperature monitoring method is provided.

Diagnostic Methods Utilizing the Composites

The provided composites disclosed herein can be used as contrast agents in diagnostic methods. Contrast agents are known for use in diagnostic methods, such as photoacoustic (PA) imaging. In particular, certain embodiments utilize a combination of PA and ultrasound (US) in order to image specimens using the provided composites as contrast agents.

Ultrasound (US) is the most common real-time imaging modality, providing multi-dimensional changes in morphology for clinical diagnosis and therapy. US can achieve better than about 100 μm resolution at several centimeters depth. Combining US detection and optical illumination is called photoacoustic (PA) imaging, which can measure tissue optical absorption while maintaining the high resolution and penetration of US imaging. Integrated US and PA imaging can deliver molecular sensitivity to ultrasound systems using biologically-targeted molecular contrast agents.

PA imaging uses two types of optical absorbers as sources of propagating ultrasound. One is natural optical absorbers in biological tissue, such as hemoglobin. The other is an external optical contrast agent, such as gold nanorods. Sensitive contrast agents can be functionalized to target specific biomolecular markers of diseased cells and provide significant clinical information about the disease at a molecular level in PA imaging. However, significant background tissue absorption, such as the blood pool, in many clinical applications limits both the sensitivity and specificity of PA molecular imaging. To overcome this problem, the unwanted background signal must be suppressed.

The provided methods use simultaneously (or near-simultaneously) generated ultrasound (US) and PA signals of a composite contrast agent is described herein to increase specific contrast based on suppressing undesired background objects. The absorption spectrum of the nanoparticles red-shifts to the near infrared range from the typical peak of distributed nanoparticles, enabling their use at depth in tissue. In certain embodiments the metal nanoparticles absorb at wavelengths between 600-1100 nm. Illuminating the composite with a pulsed laser with appropriately chosen parameters can heat the composite through optical absorption by the nanoparticles and expand the emulsion droplet to form a bubble, where a bubble is defined as a vapor or gas cavity that may or may not re-condense into a liquid state.

By delivering US pulses simultaneously or immediately after bubbles are generated, harmonic signals can be produced. Contrast can be enhanced by subtracting both US-alone and PA-alone signals from the simultaneous US+PA signal, with complete cancellation in objects in which no bubbles are generated (i.e., intrinsic background) but nonlinear residue signals in regions with nanoparticle-coated emulsion composites. Exemplary results show that the subtracted image of emulsion droplets with nanoparticles can enhance contrast by 12.8 dB compared to that of pure nanoparticles of the same concentration (e.g., 0.018 volume %), indicating that the sensitivity and specificity for molecular imaging applications can be enhanced for this composite contrast agent using non-linear processing of signals from simultaneous US+PA excitation.

In general, any modulation of either the pure ultrasound signal, the photoacoustic signal, or the combined photoacoustic/ultrasonic signal that is unique to the contrast agent can be used to enhance the specific contrast and reduce the effects of background signals on the resultant molecular image.

Potential applications of the compositions and methods of the present disclosure include all molecular imaging applications using integrated US+PA imaging, including, but not restricted to, cancer detection and characterization. In certain embodiments, the imaging methods including imaging cancer in the breast, prostate, and peripheral organs such as the testes, eye, and skin. In addition, minimally invasive procedures using catheter-based devices (e.g., IVUS-like technologies) can be applied to a wide range of cardiovascular applications, such as the identification and characterization of vulnerable plaque and the identification and characterization of the vasa vasorum.

The composite contrast agent can be conjugated with a biologic targeting agent such as an antibody or aptamer for targeted cellular imaging. The composite contrast agent would target a cell type of interest, such as a specific tumor cell, for both highly specific diagnosis plus potential therapy delivered by the same agent. The agent is then imaged using a combination of ultrasound and photoacoustic imaging, where specific contrast can be greatly enhanced using the methods described in this disclosure. This can lead to highly sensitive and highly specific identification of the targeted cell type since background photoacoustic and ultrasonic signals can be greatly suppressed. This method provides an effective approach for cancer diagnosis and management of cancer therapies.

Certain methods described herein relate to the non-linear interaction between acoustic waves generated by simultaneous photoacoustic and ultrasonic sources. However, any modulation of the PA or ultrasonic signal from the nanoparticle can be used for background suppression because background sources are not subject to this modulation. For example, the change in size and shape of the emulsion droplet due to heating by pulsed optical illumination can change the geometry of the nanoparticles on the droplet's surface, altering the plasmonic coupling between these particles and thereby modulating the optical absorption coefficient. The PA signal from the nanosystem can vary significantly from laser shot to laser shot and create a unique signature that can help suppress background PA signals.

Even if large, stable microbubbles are not created as part of droplet expansion during laser illumination, short-lived nanobubbles may be created of sufficient size to change either the PA signal or the combined PA/US signal on a shot-to-shot basis. Again, any modulation of the PA signal from the agent can be used to suppress stable background signals.

In one aspect, a method of imaging is provided. In one embodiment, the method includes the steps of providing a composite according to the embodiments disclosed herein; applying energy to the composite; and detecting a signal emitted from the composite. Exemplary embodiments of this aspect can be found in Example 2 below and the related figures.

In one embodiment, the composite is used as a contrast agent for imaging. Representative imaging techniques include ultrasound, photoacoustic, and combinations thereof. While PA and US systems are described in detail herein, it will be appreciated that the composites can be used as contrast agents in any diagnostic method known to those of skill in the art. The contrast agents in bubble form provide high nonlinearity and, therefore, yield the possibility of significant imaging enhancement when used with appropriate analytical techniques.

In one embodiment, the step of applying energy to the tissue results in a phase transition of the composite core. As discussed at length above, with regard to FIG. 8A, applying energy to the droplet 100 can be used to heat the liquid core 110 so as to expand it into a gaseous core 210 and produce a bubble 200.

In one embodiment, the applied energy is electromagnetic energy, as disclosed above. In one embodiment, the applied energy is sonic energy. In a further embodiment, the sonic energy is ultrasonic energy. As used herein, the term “ultrasonic energy” refers to the sonic signal having a frequency greater than 20 kHz. Ultrasonic energy provides a means for reducing the threshold energy and allows for the use of less energy and penetrate deeper to generate the effect.

In one embodiment, both electromagnetic and sonic energy are applied simultaneously. In a further embodiment, the electromagnetic energy impinges on the composite at substantially the same time as peak negative pressure induced by the sonic energy. Since negative pressure enhances the probability of a phase transition, coincident excitation with electromagnetic energy at this instant can produce significant changes in the contrast agent that can be exploited for both imaging and therapeutic applications.

In one embodiment, the step of detecting includes detecting a sonic signal from the composite.

In one embodiment, the method further includes a step of providing a dye with a substantially linear response to optical excitation in order to provide a dye signal. In a further embodiment, the method includes a step of subtracting the signal from the composite from the dye signal. By utilizing a dye as a comparative linear responder to the optical excitation, these embodiments allow for imaging that takes into account the nonlinear properties of the bubble 200 of the composite, as set forth in Example 2 below.

In one embodiment, the detection step includes a step of comparing the signal from at least two different light intensities. In one embodiment, the detection step includes a step of comparing the signal from at least two different sonic intensities. In one embodiment, the detection step includes a step of comparing the signal from at least two different light intensities and at least two different sonic intensities simultaneously. These comparisons aid in interpreting the data obtained, as exemplified in Example 2.

The following examples are intended to illustrate, and not limit, the embodiments disclosed herein.

### EXAMPLES

### Example 1

### Amphiphilic Gold Nanoparticle Composites

Amphiphilic gold nanoparticles are demonstrated to effectively stabilize emulsions of hexadecane in water. Nanoparticle surfactants are synthesized using a simple and scalable one-pot method that involves the sequential functionalization of particle surfaces with thiol-terminated polyethylene glycol (PEG) chains and short alkane-thiol molecules. The resulting nanoparticles are shown to be highly effective emulsifying agents due to their strong adsorption at oil-water and air-water interfaces. The original non-functionalized gold nanoparticles are unable to effectively stabilize oil-water emulsions due to their small size and low adsorption energy. Small angle x-ray scattering and electron microscopy are used to demonstrate the formation of nanoparticle-stabilized colloidosomes that are stable against coalescence and show significant shifts in plasmon resonance enhancing the near-infrared optical absorption.

### INTRODUCTION

The high adsorption energy of microparticles at the oil-water interface creates a large barrier for desorption and prevents drop coalescence due to steric repulsion from close-packed particles at an interface. The adsorption energy scales with the square of particle radius and frequently reaches values as high as 10⁷ kT for micron-sized particles. Unfortunately, the adsorption energy for nanoparticles is usually similar to the energy of thermal fluctuations and this severely limits their effectiveness as emulsion stabilizers. One way to improve the adsorption of nanoparticles to an oil-water interface is to modify the particle surface. Certain researchers have shown an improvement in emulsion stabilization by functionalizing Fe₃O₄ nanoparticles with a carboxylic acid and compared this to the adsorption of bare particles. Others have measured an increase in the binding energy of gold nanoparticles to oil-water interfaces after grafting mercaptoundecyl-tetra(ethylene glycol) to the gold surface. Another way to improve particle adsorption to an interface is to graft surface-active polymers onto the particle surface. Others have shown with simulations that nanoparticles grafted with a purely hydrophilic polymer do not adsorb to an oil-water interface while particles grafted with amphiphilic block copolymers composed of at least 30% hydrophobic polymer will adsorb to an oil-water interface with at least a 90% probability. Others have also modified iron nanoparticles by grafting a poly(methacrylic acid)-poly(methyl methacrylate)-poly(styrenesulfonate) triblock copolymer to make them effective stabilizers of dodecane in water emulsions. Similarly, they also grafted poly(2-(dimethylamino)ethyl methacrylate) onto silica nanoparticles that could be tuned to penetrate the oil-water interface to different extents. While this elegant approach of polymerizing copolymers on particle surfaces proved to be highly effective, the processes required for synthesizing tethered nanoparticles can be labor intensive and costly.

Recently, the inventors developed a simple, scalable and cost effective method for synthesizing gold nanoparticle surfactants that spontaneously self-assemble into clusters with controllable structure. In this approach, colloidal gold in water is first functionalized with thiol terminated poly ethylene glycol (PEG) through simple thiol chemistry. The long, bulky PEG-thiol chains sterically stabilize the particles in water. Subsequent functionalization with a short alkane-thiol renders the particles amphiphilic and induces short-ranged attraction. The resulting particles are surface active and form rafts at the air-water interface and stable nanoparticle clusters in dispersion. These clusters are reminiscent of micelles formed from molecular surfactants with aggregation numbers that can also be controlled by modifying the grafting density of the polymer on the nanoparticle surface. Furthermore, we speculate that the different thiol-terminated molecules phase segregate on the gold nanoparticle surface. Previous work has demonstrated that surface-bound thiolated molecules are mobile on gold surfaces and that mixtures of ligands will self-segregate. More recently, simulations have been shown that different ligands can form striped, ordered patterns on gold nanoparticle surfaces. Other work shows that these ligands can also desorb from gold surfaces and exchange positions with unbound thiol molecules rather than diffuse along the gold surface. In another recent publication we show that for the amphiphilic particles used in this study, the alkane-thiol molecules replace the previously bound PEG-thiol chains. As a result, the alkane-thiol acts as a molecular spacer and controls the distance between particles within each cluster.

Like molecular surfactants, the nanoparticle surfactants are also effective emulsifiers due to their amphiphilic nature. During the emulsification of oil in presence of these dispersions, the clusters readily break up and particles adsorb to the oil-water interface. FIG. 2 illustrates the preparation method for the nanoparticle surfactants and the hypothesized particle arrangement at the interface after emulsification. In this manuscript, we demonstrate the effectiveness of these emulsifiers through the formation of stable hexadecane in water emulsions using several types of nanoparticle surfactants. We also show the arrangement of the particles at the hexadecane-water interface is a function of the length of the alkane-thiol spacer and of the PEG-thiol surface concentration.

### Materials and Methods

Hexadecane, sodium citrate, gold chloride trihydrate, butane-thiol, octane-thiol and dodecane-thiol are purchased from Sigma Aldrich (St. Louis, Mo.) and used as received. Thiol-terminated Poly(ethylene glycol) methyl ether (10 kDa) is obtained from Polymer Source (Dorval, Quebec Canada). Colloidal gold nanoparticles are synthesized using the citrate reduction method to produce a colloidal suspension of 12 nm diameter particles in an aqueous buffer that is 0.0015 vol % particles. The particles are rendered amphiphilic by sequential functionalization with PEG-thiol and alkane-thiol using a process described in a previous publication. The surface concentrations of PEG-thiol used in this study are 0.6, 0.8 and 1.8 chains/nm² Au as determined from thermogravimetric analysis (TGA). After functionalization, the particles are concentrated by pressurized diafiltration (Millipore) to a 0.024 vol % gold dispersion.

Hexadecane-in-water emulsions (1 vol % hexadecane and 0.02 vol % gold) are prepared using a Branson Digital Sonifier with a 102C microtip operated at 30% amplitude power that is pulsed 1 second on, 1 second off for a total of 1 minute in the presence of particles. The UV-Vis spectroscopy is carried out using a Thermo Scientific Evolution 300 system in the visible and ultra-violet range (300-1100 nm). The size distribution of the emulsified oil drops is examined with a Zeiss Axiovert 40 CFL optical microscope at 40× magnification. Hydrodynamic radii of droplets are measured using Dynamic Light Scattering (DLS) with a Malvern Zetasizer Nano ZS (Worcestershire, United Kingdom) using a laser wavelength of 633 nm. The lyophilized oil droplet dispersions are also examined at a 15,000× magnification with a FEI Sirion Scanning Electron Microscope (SEM) operating at 5 kV. Freeze-dried oil droplets and clusters are also examined with a FEI Tecnai G2 F20 Transmission Electron Microscope (TEM) operating at 200 kV. Images are processed using ImageJ software developed at the National Institutes of Health. Interfacial tension measurements are performed with a Kruss Tensiometer K12 equipped with a Wilhelmy slide.

Small-Angle X-ray Scattering (SAXS) is used to determine the particle arrangement before and after emulsification. SAXS experiments are performed in an Anton Paar SAXSess instrument (Graz, Austria) with a line-collimation system using a Cu-K_{α} source with a wavelength of 1.54 Å. The data are placed on an absolute scale by referencing to the scattering of pure water which is known to have an absolute scattering of 0.0162 cm⁻¹. The scattering curves are subsequently desmeared using the Lake iterative method prior to data fitting with Igor macros developed at NIST. T smeared scattering curves are also fit directly using the DANSE SansView software and both methods result in identical fit parameters. SAXS experiments are performed on 0.005 vol % gold samples. The difference in scattering length density (SLD) of gold and water (Δρ_{Au-H2O}=1.14*10⁻⁴ Å⁻²) is much greater than hexadecane and water (Δρ_{C16H34-H2O}=1.94*10⁻⁶ Å⁻²), so the gold particles dominate the scattering.

### Results and Discussion

Amphiphilic gold particles functionalized with PEG-thiol and alkane-thiol using the new synthesis scheme are shown to readily adsorb at a macroscopic hexadecane-water interface. The particles are surface active and can reduce the interfacial tension, similar to a small molecule surfactant. We measure the interfacial tension of our hexadecane and water to be 34.72±0.32 mN/m. The interfacial tension value decreases to 28.05±0.32 mN/m when 0.8 PEG chains/nm² Au and butane-thiol functionalized particles are added at extremely low concentrations (0.00005 vol % Au). The interfacial tension is also constant at much higher particle concentrations (up to 0.005 vol % Au). In addition, the interfacial tension values are also affected by the type of alkane-thiol that coats the nanoparticle surfactants. The interfacial tension of hexadecane and water in the presence of 0.005 vol % Au particles that are functionalized with 0.8 PEG chains/nm² Au and octane-thiol or dodecane-thiol is 29.80±0.29 mN/m and 31.12±0.28 mN/m respectively. This shows that shorter alkane-thiol chains reduce interfacial tension to a larger extent, implying that they are more surface active.

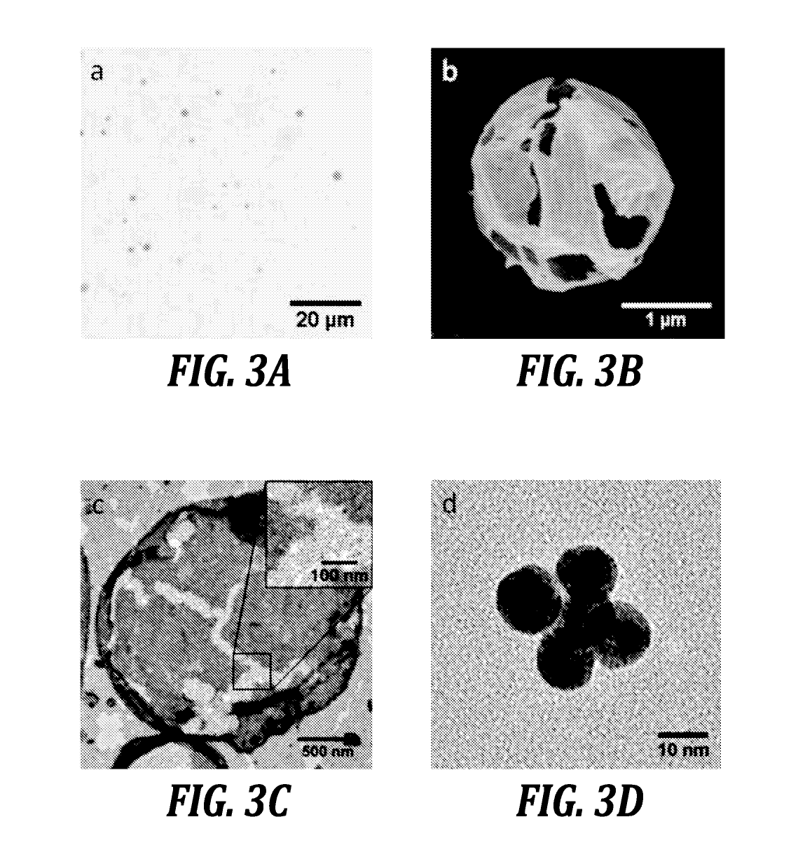

In this study, we examined Pickering emulsions composed of 1 vol % hexadecane and 0.02 vol % amphiphilic gold particle loadings in water at neutral pH. The formation of particle-stabilized oil droplets from nanoparticle surfactants containing 0.8 PEG chains/nm² Au and butane-thiol on the surface is confirmed with several microscopy techniques. Optical microscopy of a dilute emulsion shows many small stable droplets that are approximately 1 μm diameter (FIG. 3A). Smaller drops could also be present but these would not be visible with simple optical microscopy. Scanning electron microscopy (SEM) has a higher magnification that allows closer examination of nanoparticle-stabilized emulsion droplets. Unfortunately, the samples had to be dried prior to analysis with electron microscopy. Freeze-drying (lyophilizing) was used to dry the samples while preventing the complete destruction of the interfacial particle structures so that these could be imaged. In FIG. 3B we see that the droplets are partially deflated and fractured from the drying process, but the particles still retain their shell structure after the hexadecane and water have fully evaporated. Only one representative droplet is shown here, but tens of similar droplets were imaged using this approach. Finally, transmission electron microscopy (TEM) is also shown in FIG. 3C. This technique also shows collapsed droplets with parts of the shell structure still evident. However, the higher resolution of TEM also allows for imaging of the individual gold particles at the interface. TEM is also used to examine the structure of the particle clusters that form in water (analogous to surfactant micelles) before emulsification. These clusters are analyzed in the same manner as a previous publication. The clusters are lyophilized onto a TEM grid to minimize the interfacial clustering effects that can occur during the drying of dispersions. Unfortunately, many of the cluster structures collapse with the removal of solvent, but the number of particles within each cluster can still be quantified. While there is a clear distribution of cluster sizes, the most abundant cluster geometry for the 0.8 PEG chains/nm² Au and butane-thiol functionalized particles is a structure with five particles in each cluster. One of these representative clusters is also shown in FIG. 3D and additional images along with the size distribution of the clusters are located in the Supplemental Information.

Spectroscopy gives further information about the relative particle arrangement in the dispersed emulsions. Colloidal gold exhibits unique absorbance spectra that are dependent on the particle size and shape due to surface plasmon resonance. The characteristic peak for 12 nm bare gold particles occurs at 520 nm and this is red-shifted when particles are in close proximity to each other. FIG. 4A shows UV-Vis absorbance measurements for PEG-functionalized particles before (individual particles) and after (multi-particle clusters) butane-thiol functionalization and for hexadecane emulsions stabilized with the nanoparticle surfactants. The peak absorbance for particles with 0.8 PEG chains/nm² Au prior to butane-thiol addition occurs at a wavelength of 522 nm and appears red in color as shown in FIG. 4B. The slight shift in absorbance peak occurs because of a change in the local refractive index around the particles due to the bound PEG chains surrounding the particles. After the addition of butane-thiol, the particles are amphiphilic and the absorbance peak shifts more significantly to 536 nm and the dispersion now appears purple. This occurs because the particles are organized into small clusters and the plasmon resonance of particles couples with neighbors within the cluster to shift the resonant wavelength. Finally, the absorbance peak broadens and shifts dramatically to 680 nm when the particles are emulsified with hexadecane, indicating that the particles are close-packed and form a dense shell at the emulsion interface. FIG. 4C shows a picture of the concentrated emulsion with light illuminating the sample from behind (transmitted) and from the front (reflected). This demonstrates the deep blue color acquired by the emulsion due to increased absorption of red light and also the metallic sheen that is a clear indication that particles are close-packed at the oil-water interface.

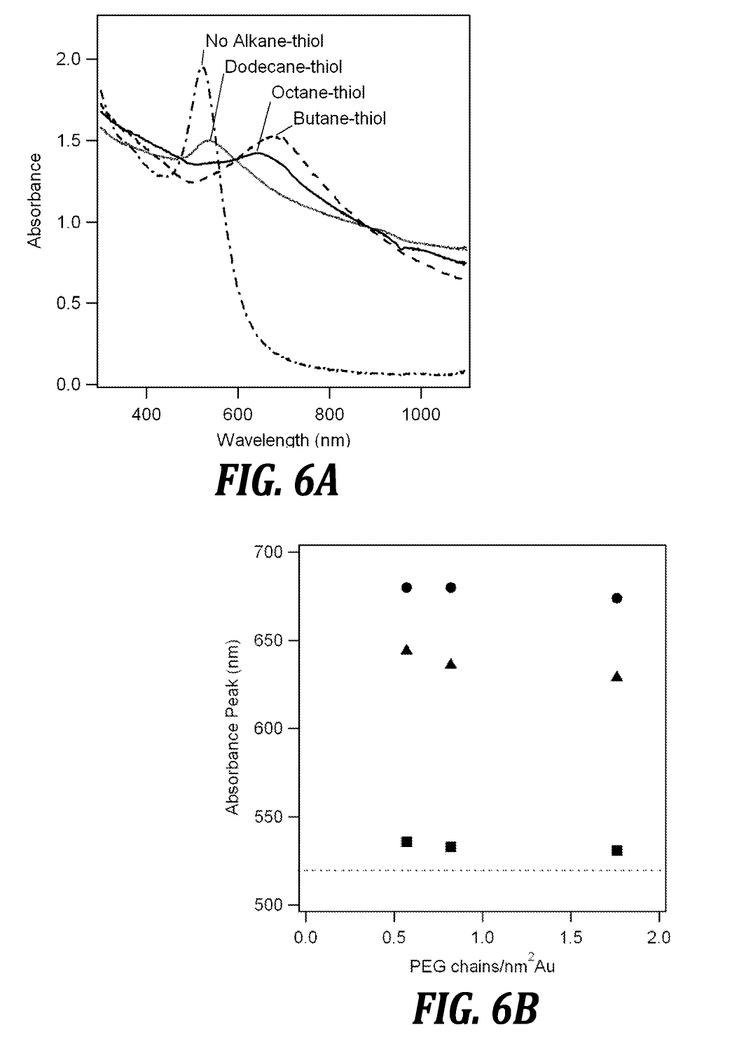

Nanoparticle surfactants composed of different PEG-thiol concentrations and different alkane-thiols will also effectively stabilize emulsions but to different extents. In addition to inducing adsorption, the length of the alkane-thiol and the surface PEG-thiol concentration also control how closely the particles are able to pack when located at the interface. FIG. 6A shows the absorbance spectra for emulsions stabilized by particles with 0.6 PEG chains/nm² Au and different alkane-thiols. It is clear that, when the length of the alkane-thiol shrinks, the peak absorbance red-shifts to higher wavelengths. This indicates that the particles are more closely packed at the interface allowing for stronger coupling between surface plasmons in adjacent particles. A slight red-shift of absorbance peak is also observed for the particle clusters prior to emulsification, however this is much smaller than the change that is observed for the emulsions. Spectral data for particle clusters prior to emulsification is located in the Supplemental Information. Particles containing PEG-thiol but without alkane-thiol are not able to stabilize an emulsion, and the spectrum is identical to that of the particles in water prior to emulsification. Although the samples were initially turbid, the samples recovered the original spectra after the emulsion droplets coalesced. FIG. 6B shows the wavelength of the peak absorbance plotted as a function of the surface concentration of PEG-thiol and the type of alkane-thiol. For all PEG-thiol loadings, emulsions using the butane-thiol particles (circles) have the largest shift in absorbance wavelength while the dodecane-thiol particles (squares) result in emulsion with the smallest shift. The PEG-thiol concentration in the nanoparticle surface also causes slight changes in the absorbance where higher amounts result in a smaller peak shift due to larger steric interactions between neighboring particles.

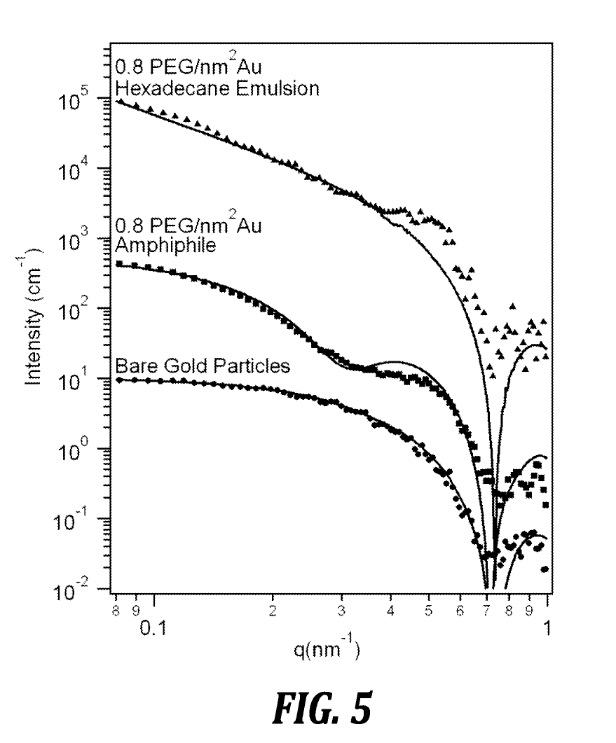

SAXS allows us to directly quantify particle adsorption at the oil droplet interface and values are found to be quite high. The scattering curves for all samples, containing different polymer concentrations and alkane-thiol lengths, are also fit using the Pickering emulsion model as demonstrated in FIG. 5.

FIG. 5 presents desmeared SAXS data for bare gold nanoparticles, clustered amphiphilic gold nanoparticles in water and emulsified amphiphilic gold nanoparticles. The bottom curve is scattering from bare gold particles in water which is fit by a spherical model with Rₚ=6.11 nm, φₚ=0.00024 ρ_{w}=9.46*10⁻⁶ Å⁻² and ρₚ=1.25*10⁻⁴ Å⁻². The middle curve (shifted by a factor of 10 for clarity) is the scattering data for a nanoparticle surfactant cluster in water and the modeled scattering curve calculated using the Debye equation. The top curve (shifted by a factor of 10⁴) is for a 1.0 vol % hexadecane in water emulsion stabilized by 0.024 vol % 6.11 nm amphiphilic gold particles. Parameters of the model are Rₒ=494 nm with a polydispersity index of 0.1 (Gaussian distribution), Rₚ=6.11 nm, δ=0, φₒ=0.00998, φₚ^{T}=0.00024, ρ_{w}=9.46*10⁻⁶ Å⁻², ρₒ=7.52*10⁻⁶ Å⁻², ρₚ=1.25*10⁻⁴ Å⁻², 88.0% interface coverage.

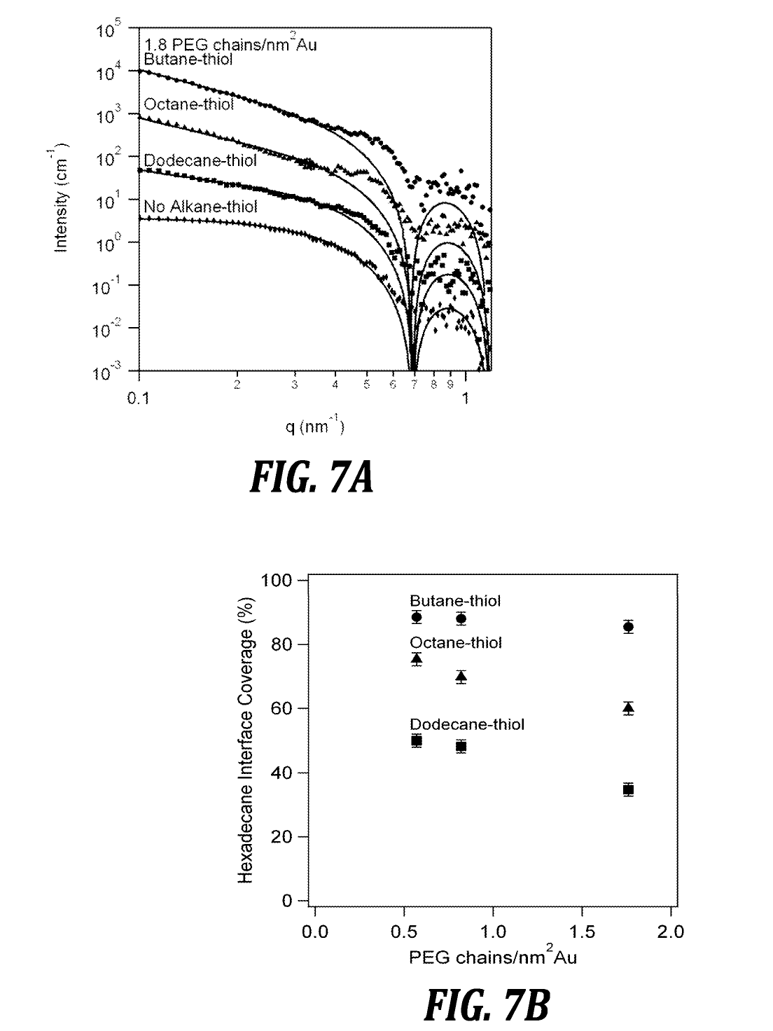

The scattering length densities and oil and particle volume fractions are held fixed as before, but the particle radii are 6.55 nm for the 0.6 and 1.8 PEG chains/nm² Au nanoparticle surfactants, because a different batch of particles was used. All fits result in an oil droplet radius of 498±2 nm and a polydispersity index of 0.1 (Gaussian distribution) but with varying nanoparticle surface coverage. Examples of the scattering curves and data fits are shown in FIG. 7A for the 1.8 PEG chains/nm² Au particle emulsions. The increasing slope with decreasing alkane-thiol length at low q indicates that more particles cover the oil-water interface for shorter thiols. We also clearly see that particles with PEG-thiol but without alkane-thiol functionalization do not show any interfacial particle adsorption and the emulsion is therefore unstable. The percentage of the oil surface area that is covered with particles is plotted as a function of PEG-thiol concentration for various nanoparticle surfactants in FIG. 7B. The particles with 0.6 PEG chains/nm² Au and butane-thiol result in the highest interface coverage (88.5%) at a hexadecane-water interface, which nearly reaches the limit of hexagonally closed-packed particles (90%). Using particles with the same PEG-thiol loadings but with octane-thiol or dodecane-thiol, causes the percent interface coverage to reduce to 75.4% and 50.0% respectively. This corresponds to an increase in the average gold particle spacing of 0.5 nm and 2.1 nm relative to emulsions stabilized with butane-thiol. The measured difference between butane-thiol monolayer film thicknesses on flat substrates and those of the longer alkane-thiols is approximately 0.5 nm (octane-thiol) and 1.3 nm (dodecane-thiol). This correlates well with the spacing differences calculated from scattering suggesting that the small thiol ligands are acting as spacers between particles. However, this trend breaks down at higher PEG-thiol loadings. Particles with 1.8 PEG chains/nm² Au and dodecane-thiol adsorb strongly to the interface but have a significantly lower interface coverage value (34.6%). This suggests that these surfactant nanoparticles are effective emulsifiers, but the gold only covers a small area of the total interface and therefore needs fewer particles to stabilize the emulsion.

We also observe a clear correlation between the packing density measured with SAXS and the absorbance shift in the spectroscopy data. Emulsions formed with butane-thiol nanoparticle surfactants have the highest particle packing at the interface and this results in a spectrum where the main plasmon peak is red-shifted by the largest extent. The butane-thiol nanoparticle surfactants have the most efficient packing because they have the shortest alkane chain length. Additionally, we can tune the packing at the interface by controlling steric hindrance through variations in the PEG-thiol concentration. By modifying the inter-particle spacing of particles at the interface, we can thus tune the UV-Vis absorption spectra. This is particularly important in emerging applications such as photothermal therapy where we must carefully engineer absorbance in the NIR region for optimal heating.

Bare gold nanoparticles and PEG-functionalized particles without alkane-thiol do not adsorb at the oil-water interface nor do they exhibit any emulsification properties. The UV-Vis spectra and x-ray scattering curves are identical before and after emulsification in the presence of bare particles and no stable oil droplets are observed with microscopy. Conversely, the data clearly shows that the nanoparticle surfactants are stabilizing the hexadecane in water emulsion and they are adsorbed at the oil-water interface. This is significant because it indicates that the self-assembly of particles at an interface can be tuned through a simple functionalization protocol to form nanoparticle surfactants. Particle adsorption at oil-water interfaces has previously been demonstrated using particles grafted with amphiphilic block copolymers. Our approach, however, does not require costly or time-consuming polymerization and purification steps. Furthermore, the particle packing density can be tuned through the formulation of different types of nanoparticle surfactants.

The ability to tune nanoparticle packing at a dispersed oil interface and also the resulting plasmon resonance of metallic particles can be useful for a number of applications. As previously discussed, the ability to engineer the gold particle spacing and therefore the NIR absorbance is critical to the design of nanostructures for photothermal therapy. While solid gold shells have been proven effective theranostic agents due to a high NIR absorbance, they are too large to clear through the renal system. Thus, composite structures of small nanoparticles (D<5 nm) that are NIR absorbent and can be cleared by the renal system could be advantageous for new cancer therapies. A tunable plasmon resonance can also be utilized in the design of organic photovoltaic devices. Recently, Wu et al. showed an improvement in organic solar cell efficiency by utilizing the plasmonic effects of gold nanoparticles that were incorporated into the polymer network. Utilizing a self-assembling structure with a tunable plasmon resonance might result in even further enhancements in device efficiency. For this application, the photoactive polymer could be encapsulated in the self-assembled gold microstructure prior to coating in order to harness more energy from the photovoltaic device. The vastly different applications briefly discussed here illustrate the potential impact that these self-assembling nanoparticle surfactants can have in the development of new nanomaterials. The ability to tune inter-particle spacings and the spectroscopic absorbance are critical to the design of useful plasmonic nanostructures. Furthermore, this simple technique could also be extended to promote the interfacial organization of quantum dots and other sophisticated nanoparticle systems.

### CONCLUSION

Self-assembling nanoparticle surfactants are shown to effectively stabilize hexadecane in water emulsions. These functional nanoparticles are synthesized through sequential grafting of controlled amounts of hydrophilic PEG-thiol chains and short hydrophobic alkane chains using thiol chemistry. This simple protocol results in amphiphilic particles that are able to assemble at an oil-water interface at high interfacial concentrations. Furthermore, it is possible to control the particle packing density at the interface through manipulation of steric interactions. This also provides a mechanism to tune the plasmon resonance of the self-assembled particles. These new types of particles make it possible to design new nanomaterials for a wide range of applications in nanomedicine and renewable energy, among others.

### Example 2

### Simultaneous Ultrasound and Photoacoustic Analysis Using Nanoparticle Composites

The present example utilizes the composites of the disclosed nanoparticle composites as contrast agents for use with ultrasound (US) imaging, photoacoustic (PA) imaging, and combinations thereof (PA+US).

FIG. 8B shows an example of an experimental setup for stimulating and imaging the composite contrast agents of the present disclosure, including a tube containing the samples, a pulsed optical source, a linear array transducer for delivering US pulses, and a wideband PVDF (polyvinylidene fluoride) transducer to receive the acoustic signals.

The PTFE (Teflon) tube (SLTT-16-72, Zeus, WA) with an inner diameter of 1.6 mm and a thickness of 38 μm was positioned in a tank filled with DI water. The tube was filled with the samples to be tested.

The optical source was providing by a wavelength tunable OPO system (Surelite OPO plus, Continuum, Santa Clara, Calif.) pumped by a frequency-doubled pulsed YAG laser (Surelite 1-20, Continuum, Santa Clara, Calif.) delivered 5 ns pulses with a repetition rate of 20 Hz. The wavelength was 810 nm, within the broad plateau in the absorption spectra of the emulsion samples. The optical sources could also provide light between 600 nm and 1100 nm (the typical “therapeutics window”) at pulse lengths ranging from 1-100 nsec and pulse repetition rates ranging from 1 Hz to 100 kHz. The light was coupled into a 3.2 mm diameter fiber bundle to irradiate the tube at the tilted angle of about 60 degrees to the vertical line, resulting in a round irradiation region of about 10 mm in diameter. The fluence was estimated at 8 mJ/cm².

The linear array transducer (AT8L12-5 50 mm, Broadsound, Taiwan) interfaced with an US imaging system (Verasonics, WA) transmitted US pulses with a center frequency of 9 MHz. Half of the aperture (25 mm, 128 elements) were used to focus the beam on the tube at a 20 mm distance with a tilted angle of 30 degrees to the vertical line. A field-programmable gate array (FPGA) was used to synchronize the laser and Verasonics system to let the US pulses arrive at the sample less than 0.1 μsec after the laser pulses.

The receiving transducer was home-made with a 28 μm PVDF film, resulting in a broad acoustic band from 50 KHz to 30 MHz. The length of the active element was about 8.4 mm and the cylindrical focus was at 8 mm, forming a focusing angle of 60 degrees. The transducer was driven by a linear actuator motor (T-LA60A, Zaber, BC, Canada) to scan along the tube with a scan step of 0.2 mm. At each step, 20 acoustic signals (US, PA, or simultaneous PA/US) were averaged.

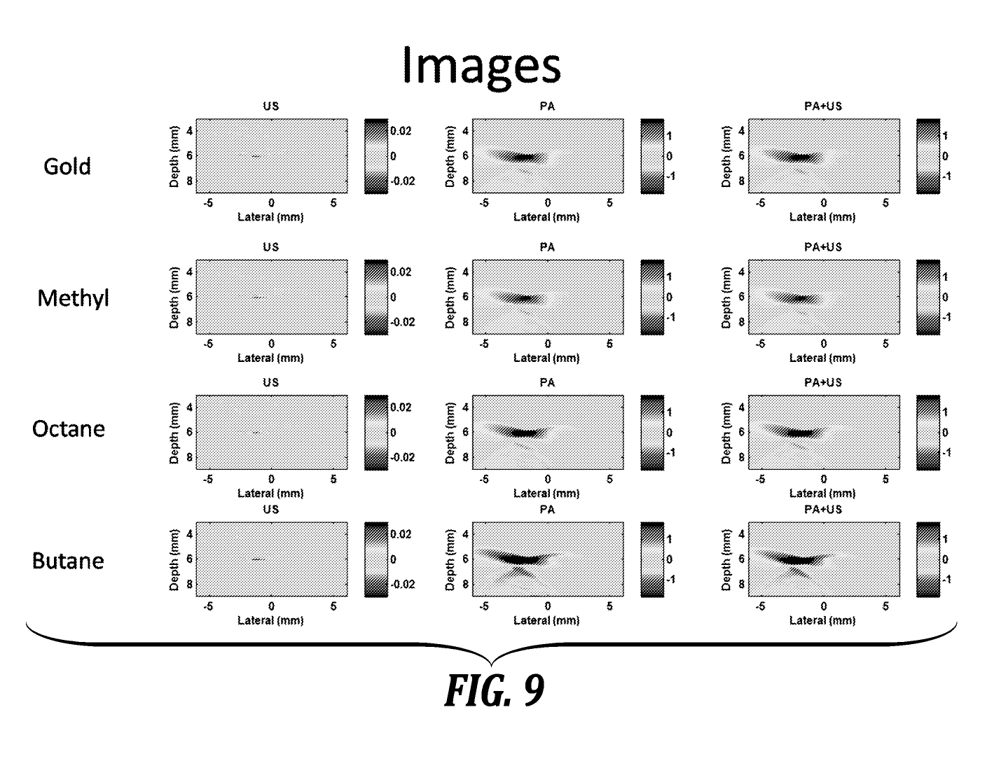

FIG. 9 presents experimental results for four different composite contrast agents (four rows). The left column presents images for US-alone probing. The central column presents images for optical probing alone (PA). The right column shows images for simultaneous US+PA probing of the system. The first row is for only gold nanoparticles not integrated into an emulsion—the response here should be nearly identical to stable background signals encountered in the body. The remaining three rows are for emulsions composed of a perfluorohexane oil core having an average diameter of about 200 nm encapsulated with gold nanoparticles having an average diameter of about 10 nm with different surface coatings and optical absorption.

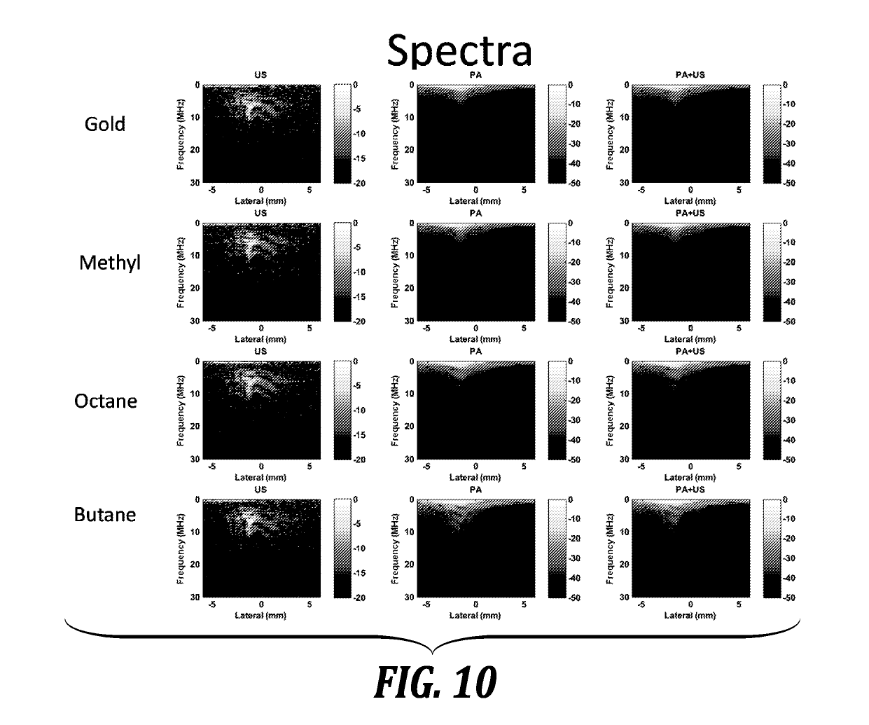

FIG. 10 presents acoustic spectra for all the images of FIG. 9. Notice the broader spectrum for the butane system suggesting the presence of non-linearities.

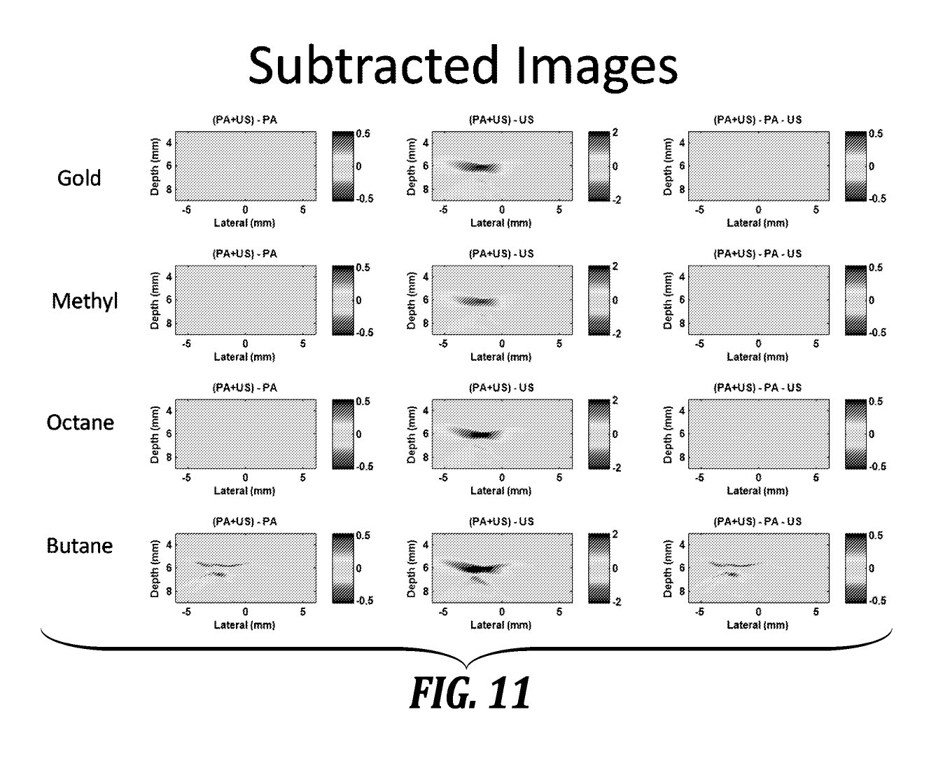

FIG. 11 presents a set of subtracted images computed from the raw images of FIG. 9. Note the strong signal for the (US+PA)-US−PA image for the butane system. This suggests either a strong non-linear response or a modulation of the PA source for this nanosystem. In either case, this signal can be easily distinguished from background sources that will act more like the pure gold system shown in the top row. These results demonstrate that the composite nanosystem combining an emulsion droplet core with, for example, gold nanospheres at the interface can be used to enhance the specific contrast of molecular images obtained by processing integrated US+PA probes.

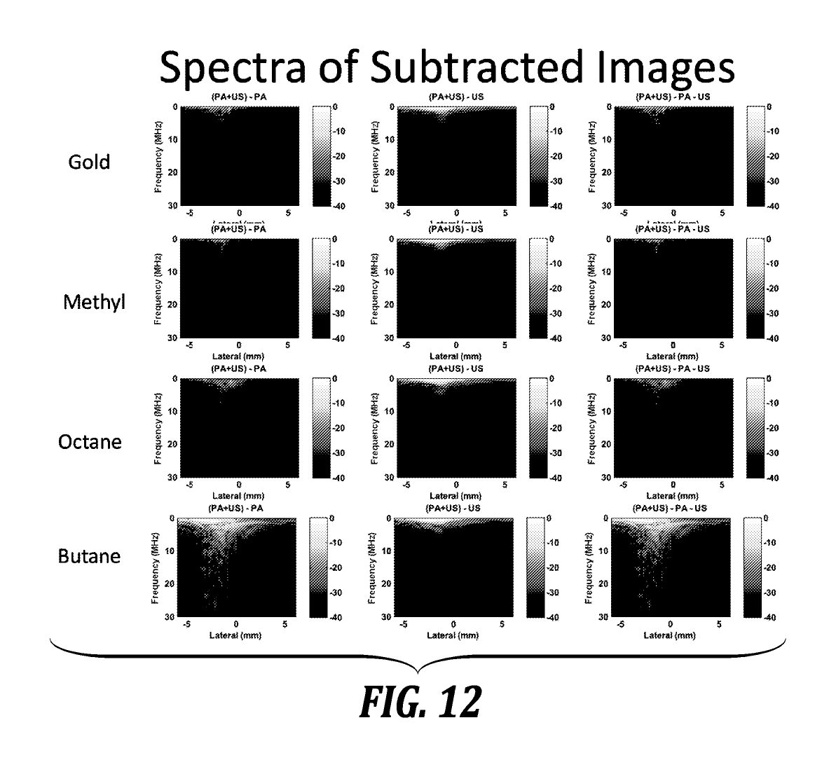

FIG. 12 shows the spectra of the subtracted images of FIG. 11. The broader spectra of the subtracted signals of the butane system can be clearly seen. By choosing a higher sub-band (1.5 MHz to 20 MHz), the contrast of the subtracted image of emulsion droplets with gold nanospheres (GNSs) over pure GNSs can increase from 12.8 dB (full band, 0.05-30 MHz) to 15.7 dB. Again, the enhanced contrast using sub-band imaging provides the evidence of generation of non-linear signals of the emulsion droplets.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

All of the references cited herein are incorporated by reference. Aspects of the disclosure can be modified, if necessary, to employ the systems, functions, and concepts of the above references and applications to provide yet further embodiments of the disclosure. These and other changes can be made to the disclosure in light of the detailed description.

Specific elements of any foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

While illustrative embodiments have been illustrated and described, it will be appreciated that various changes can be made therein without departing from the spirit and scope of the invention.

1. An emulsion, comprising:
a hydrophilic continuous phase; and
a hydrophobic dispersed phase comprising a plurality of droplets, the droplets comprising a composite, comprising:
a core comprising a hydrophobic liquid;
and a plurality of nanoparticles substantially encapsulating the core, wherein the plurality of nanoparticles are associated with a plurality of emulsifier molecules.

2. The composite of claim 1, wherein the emulsifier molecules render the nanoparticles amphiphilic.

3. The composite of claim 1, wherein the emulsifier molecules are amphiphilic molecules.

4. The composite of claim 1, wherein the emulsifier molecules comprise both hydrophobic molecules and hydrophilic molecules.

5. The composite of claim 1, wherein the emulsifier molecules are not polymers.

6. The composite of claim 1, wherein the nanoparticles are selected from the group consisting of metal nanoparticles, magnetic nanoparticles, ferroelectric nanoparticles, and semiconductor nanoparticles.

7. The composite of claim 6, wherein the metal nanoparticles comprise a metal selected from the group consisting of Gold, Silver, Copper, Palladium, and Platinum.

8. The composite of claim 1, wherein the emulsifier molecules are surfactant molecules.

9. The composite of claim 1, wherein the emulsifier molecules are selected from the group consisting of thiol-terminated polyethylene glycols, thiol-terminated straight chain alkyl molecules, thiol-terminated branched alkyl molecules, thiol-terminated perfluorocarbons, and combinations thereof.

10. The composite of claim 1, wherein the emulsifier molecules are charged molecules.

11. The composite of claim 10, wherein the charged molecules are selected from the group consisting of mercaptoundecyl-trimethylammonium bromide, 6-Amino-1-hexanethiol hydrochloride, 11-Aminoundecanethiol hydrochloride, (3-Mercaptopropyl)ammonium chloride, cysteamine, and thiol-terminated cationic peptides.

12. The composite of claim 1, wherein the emulsifier molecules are covalently bound to the nanoparticles.

13. The composite of claim 1, wherein the nanoparticles have an average diameter of from 3 nm to 20 nm.

14. The composite of claim 1, wherein the nanoparticles comprise a biologic targeting agent.

15. The composite of claim 14, wherein the biologic targeting agent is selected from the group consisting of an antibody, an aptamer, a peptide, and a protein.

16. The composite of claim 1, wherein the core has an average diameter of from 5 to 1000 nm.

17. The composite of claim 1, wherein the hydrophobic liquid of the core comprises a liquid selected from the group of hydrocarbon oils, fluorinated oils, and combinations thereof.

18. The composite of claim 17, wherein the hydrophobic liquid is a hydrocarbon oil selected from the group consisting of hexane, dodecane, cyclopentane, cyclohexane, and hexadecane.

19. The composite of claim 17, wherein the hydrophobic liquid is a fluorinated oil selected from the group consisting of perfluoropentane, perfluorohexane, perfluoroheptane, perfluorodecalin, and perfluoro(methylcyclohexane).

20. The composite of claim 1, wherein the hydrophobic liquid is capable of vaporization when heated to a temperature from 20° C. to 80° C.

21. The composite of claim 1, wherein the nanoparticles are configured to heat the core when irradiated with electromagnetic radiation.

22. The composite of claim 21, wherein the electromagnetic radiation has a wavelength of from 500 nm to 1500 nm.

23. The composite of claim 1, wherein the individual nanoparticles within the plurality of nanoparticles have an individual absorption peak and the plurality of nanoparticles have a collective absorption peak that is at a longer wavelength than the individual absorption peak.

24. The composite of claim 1, wherein each of the plurality of nanoparticles are separated from adjacent nanoparticles by a distance that is less than two times the diameter of the nanoparticles.

25. The composite of claim 1, wherein each of the plurality of nanoparticles are separated from adjacent nanoparticles by a distance that is less than two nanometers.

26. The composite of claim 1, wherein the dispersed phase maintains structural integrity when the hydrophobic liquid changes states to a gas.

27. A method of therapy comprising:
contacting a biological tissue with a composite according to claim 1; and
applying energy to the biological tissue.

28. The method of claim 27, wherein applying energy to the tissue results in a phase transition of the core.

29. The method of claim 28, wherein the phase change is from a liquid phase to a gas phase.

30. The method of claim 28, wherein the phase change ablates or otherwise destroys the biological tissue.

31. The method of claim 28, wherein the hydrophobic liquid returns to the liquid phase after the energy is no longer applied to the biological tissue.

32. The method of claim 28, wherein applying energy the biological tissue causes the composite to substantially break apart.

33. The method of claim 27, wherein applying energy to the tissue results in thermally exciting the plurality of nanoparticles.

34. The method of claim 27, wherein the applied energy is electromagnetic energy.

35. The method of claim 27, wherein the applied energy is sonic energy.

36. The method of claim 27, wherein electromagnetic energy and sonic energy are applied simultaneously.

37. The method of claim 36, wherein the electromagnetic energy impinges on the biological tissue at substantially the same time as peak negative pressure induced by the sonic energy.

38. The method of claim 27, wherein applying energy to the biological tissue comprises:
applying electromagnetic energy at a first time sufficient to cause a phase change in the core; and
applying sonic energy at a second time sufficient to urge the composite through the biological tissue.

39. The method of claim 27, wherein the biological tissue is selected from the group consisting of blood clots, atherosclerotic plaques, tumors, fat cells, fibroids, and moles.

40. A method of imaging comprising:
among a composite according to claim 1;
applying energy to the composite; and
detecting a signal emitted from the composite.

41. The method of claim 40, wherein applying energy to the tissue results in a phase transition of the composite core.

42. The method of claim 40, wherein the applied energy is electromagnetic energy.

43. The method of claim 40, wherein the applied energy is sonic energy.

44. The method of claim 43, wherein the sonic energy is ultrasonic energy.

45. The method of claim 40, wherein electromagnetic and sonic energy are applied simultaneously.

46. The method of claim 45, wherein the electromagnetic energy impinges on the composite at substantially the same time as peak negative pressure induced by the sonic energy.

47. The method of claim 40, wherein detecting comprises detecting a sonic signal from the composite.

48. The method of claim 40, furthering comprising providing a dye with a substantially linear response to optical excitation in order to provide a dye signal.

49. The method of claim 48, furthering comprising subtracting the signal from the composite from the dye signal.

50. The method of claim 40, wherein detection comprises comparing the signal from at least two different light intensities.

51. The method of claim 40, wherein detection comprises comparing the signal from at least two different sonic intensities.

52. The method of claim 40, wherein detection comprises comparing the signal from at least two different light intensities and at least two different sonic intensities simultaneously.


SEQUENCE LISTING (1 sequence)

SEQ ID NO: 1 - DNA, 11 bp, Artificial
  Synthetic
aaaaakkkkkk